(19) United States
(12) Patent Application Publication
(10) Pub. No.: US 2021/0022593 A1
(43) Pub. Date: Jan. 28, 2021

(54) Photoplethysmography Imaging (PPGI)-Based Pulp Vitality Test

(71) Applicant: UNITED STATES OF AMERICA AS REP BY SEC OF THE NAVY, SILVER SPRING, MD (US)

(72) Inventors: ROY DORY, San Antonio, TX (US); JUSTIN P. BEQUETTE, SAN ANTONIO, TX (US); SENAY TEWOLDE, DALLAS, TX (US)

(73) Assignee: UNITED STATES OF AMERICA AS REP BY SEC OF THE NAVY, SILVER SPRING, MD (US)

(21) Appl. No.: 16/937,899

(22) Filed: Jul. 24, 2020

Related U.S. Application Data
(60) Provisional application No. 62/878,087, filed on Jul. 24, 2019.

(51) Int. Cl. A61B 1/24; A61B 5/1455; A61B 5/026; A61B 5/0205; A61B 5/00; A61B 1/00; A61B 5/0402; A61B 1/06

(52) U.S. Cl. CPC A61B 1/24; A61B 5/14551; A61B 5/0261; A61B 5/0205; A61B 5/02427; A61B 1/00045; A61B 5/0402; A61B 1/06; A61B 5/721

(57) ABSTRACT

This invention discloses a vitality-based photoplethysmography imaging (PPGI) system capable of determining the pulse frequency within the dental pulp, allowing for direct, accurate, real-time visualization of tooth vitality. The system comprises a transilluminating bitewing, capable of aligning and stabilizing a light source and an intraoral camera, which is operationally connected to a computing device that is equipped with video stabilization and digital signal processing algorithm (Pulp Assessment by Local Observation, i.e. PABLO) to assess pulp vitality via analyzing videos captured with the intraoral camera.

### CROSS-REFERENCE TO RELATED APPLICATIONS

This application claims priority to U.S. Provisional Application 62/878,087, filed on Jul. 24, 2019.

### TECHNICAL FIELD

This invention is directed to a photoplethysmography imaging (PPGI)-based pulp vitality testing system, and the method using said system to assess the distribution of perfused endodontic tissues and evaluating the health of the pulp.

### BACKGROUND

Endodontic maladies, which affect the pulp of the tooth, can be particularly difficult to diagnose. A review of U.S. Marine Corps dental records between 2003 and 2006 found nearly 20% of in-theater dental emergencies were classified as endodontic, and approximately 49% of those emergencies were not predicted in previous oral examinations [2]. The development of an improved diagnostic test to assess the status of the pulp could enable dentists to better predict tooth health and inform treatment decisions prior to deployment, thereby reducing costs incurred as a result of endodontic dental emergencies.

Vitality testing is an important aid in the diagnosis of pulp disease and apical periodontitis. Thermal and electric pulp sensibility tests, the current gold standard, have been used to indirectly determine the state of pulpal health by assessing the condition of the nerves within the dental pulp. Although these traditional tests offer high sensitivity, the response does not provide any information about the state of the pulp tissue, often generating false positive or negative results. To resolve this problem, radiographs are often administered in conjunction with sensibility tests. However, radiographs only provide images of the denser enamel, and dentin regions of the tooth, showing defects in the hard-tissue surrounding the pulp, but providing limited diagnostic utility for assessing the state of the soft pulp tissue. Additionally, radiographs are costly, often requiring specialized equipment, and dedicated resources to operate and maintain.

Recent research efforts have explored the use of Laser Doppler flowmetry (LDF) and pulse oximetry to directly assess pulp vitality. LDF, a microvascular blood perfusion assessment technique, measures slight shifts in the wavelengths of incident, and reflected light scattered by moving red blood cells. Studies have shown that LDF laser light can be transmitted through tooth enamel and dentin, and detect blood flow within the pulp vasculature. However, the technique is susceptible to noise contamination from light reflected off periodontal tissues, and any movement of the LDF sensor relative to the tooth also changes the measured wavelength shift, producing an artifact in the measurement. Additionally, LDF is highly susceptible to external sources of error, such as interference of the blood flow in the gingiva, and obstruction of the light pathway.

Pulse oximetry, which is ubiquitous in the clinical setting, uses a similar approach to LDF, but is relatively less expensive and provides a more stable measurement of regional perfusion. Pulse oximetry measures differences between the intensity of light absorbed by perfused tissue, at multiple wavelengths, to derive blood oxygenation. The amount of light blood absorbed at certain visible-red and near-infrared wavelengths fluctuates with the pulse as the proportion of oxygen-rich arterial blood changes. Unlike LDF, the light source and the detector can be positioned on the opposing sides of the perfused tissue, and by measuring light at only the specific wavelengths of interest, pulse oximetry can provide a more robust measurement less affected by sensor motion. A photodetector identifies absorbance peaks caused by pulsatile blood circulation, and thereby calculates the pulse rate and oxygen saturation level. Recent studies have shown that modified, commercially-available, finger pulse oximeters can measure blood oxygenation within the pulp vasculature, and can differentiate between vital and root-filled teeth. However, standard pulse oximeters are susceptible to noise contamination from external sources, including blood flow in periodontal tissues, such as the gingiva.

Camera-based tools have recently been investigated to image blood oxygenation in soft tissues, including vasculature in the retina, and regions of ischemic tissue in diabetic wounds. It is the goal of this invention to develop a photoplethysmography imaging based diagnostic tool for dental applications, in particular, imaging the distribution of perfused pulpal tissue, and assessing pulp vitality. This assessment could allow dental professionals to draw important conclusions about dental tissue status, including vitality and the presence of a range of endodontic maladies.

### SUMMARY OF INVENTION

In photoplethysmography imaging (PPGI)-based pulp vitality system of this invention, the image capturing device serves as an array of individual sensors to provide a spatially resolute map of tissue perfusion. The 2-dimensional spatial array allows the test to differentiate regions of interest from external sources of error. Each pixel of the digitized image serves as a discrete signal at a discrete location, together providing a spatial representation of blood oxygenation of perfused pulpal tissues. Light passing through perfused tissues generates a pulsatile signal, while light passing through non-perfused tissues remains static. By analyzing the strength of the pulsatile signals at each pixel, blood-perfused regions of tissue can be spatially differentiated from those that do not receive blood flow, allowing direct assessment of the pulpal tissue.

### DESCRIPTION OF FIGURES

A clearer understanding of the present invention can be achieved by studying the following detailed description of the preferred exemplary embodiment together with the drawings in which:

FIG. 1 shows an exploded view of the imaging hardware comprising of a transilluminating bitewing and light source housing.

FIG. 2 shows an exploded view of the imaging hardware comprising of the transilluminating bitewing with light source housing and its components.

FIG. 3 shows a schematic of the bitewing when placed inside the oral cavity next to the teeth of interest.

FIG. 4 shows a schematic of the inventive PPGI system, which includes the imaging hardware, intraoral camera, and display of interface.

FIG. 5 shows data obtained from ex-vivo testing.

FIG. 6 shows the steps of steps of pulse rate estimation from pulp vitality testing using the PABLO algorithm.

### DETAILED DESCRIPTION OF THE INVENTION

### Definition

“Transilluminate” is referred to as allowing light to pass through a sample or part of the body. It is used in this case for the tooth, and parts of the tooth such as dentin, enamel, and the pulpal tissue.

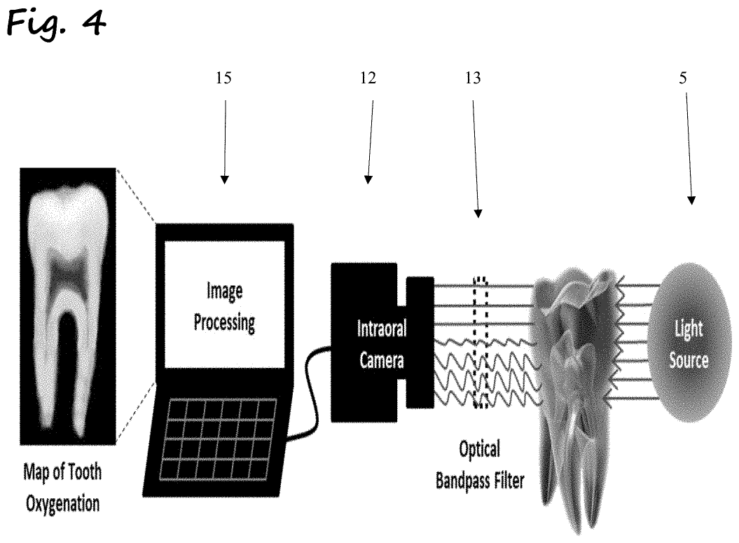

A novel PPGI-pulp vitality testing system of this invention is developed to generate a distribution map of perfused pulpal tissues, and thereby determine the vitality of a dental pulp (FIG. 4). The system comprise a light source (5), a bitewing (4) and an image capture device (12) such as intraoral camera. The image capturing device (12) is further operationally connected to a computing device (15), which is equipped with an image processing software. Additional lenses or filters (13) may be placed in front of the image capturing device (12) to reduce artifacts and noise. Each image frame of the captured video of tooth represents discrete signals at discrete locations of the tooth. Over a series of sequential image frames (i.e. a video), light passing through perfused tissues generates a pulsatile signal, while light passing through non-perfused tissues remains static. By analyzing the strength of the pulsatile signals at each pixel, blood-perfused regions of tissue can be spatially differentiated from those that do not receive blood flow, allowing direct assessment of the vitality of pulpal tissue.

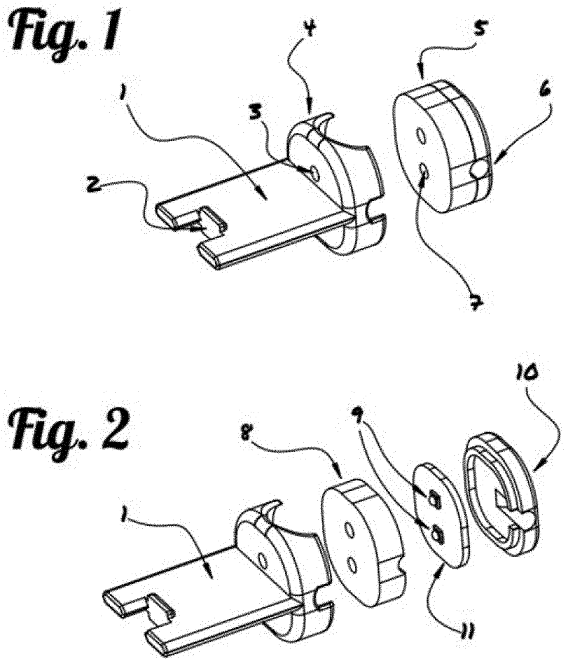

Illustration of the imaging hardware is shown in FIG. 1 and FIG. 2. According to FIG. 1, the imaging hardware of the inventive system, comprises a light source (5), a bitewing (1), which connects the light source to a image capture device (12, shown in FIG. 4), and stabilizes the light source (5) and the image capture device (12) located on the opposite sides of the target tooth.

The light source (5) is designed to transilluminate the tooth. This light source can be set to emit light within the visible or near-infrared spectrum. In one embodiment as shown in FIG. 2, the light source (5) further comprises, a pair of light source housing halves (8, 10), which when fastened to each other, creates a housing that encloses two LEDs (9), which are affixed to a circuit board (11). The circuit board adapted to fit within the light source housing, such that each LED is aligned with an opening (7) in the front half of the housing (8), directing LED light away from the front of the housing. The light source (5) may be powered by either a wired electric source, such as via USB cable (6), or by a battery, which may be placed the circuit board (11).

A bitewing (1), is used to stabilize and align the light source (5) with the image capturing device (12), such as the intraoral camera (not shown in FIG. 1), as well as maintain the proper distance between the imaging capturing device (12) and the light source (5). In one embodiment, the bitewing (1) comprises a bite surface, and a distal and proximal end. A housing receiver (4) is placed on the distal of the bitewing (1), which is designed to receive and hold the light source (5) in place such that the two openings (3) on the housing receiver (4) aligns with the opening front half of the housing (8), as to allow the passage of LED light to the target tooth. A groove (2) is made on the periphery of the proximal end of the bite wing (1), which is designed to receive, position, align and stabilize the imaging capturing device (12) in relation to the light source (5).

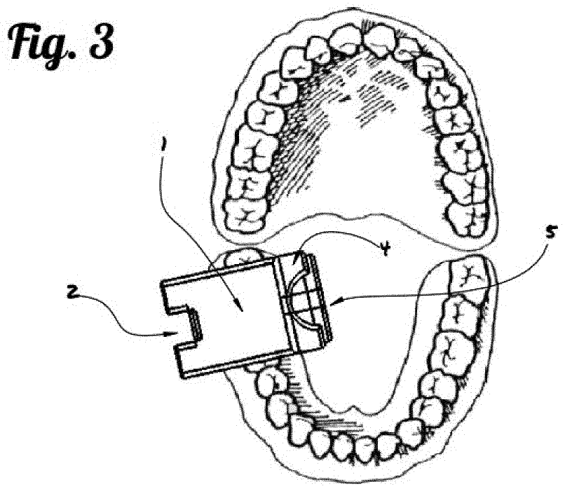

During operation as shown in FIG. 3, the light source (5) is inserted into the housing receiver (4) on the bitewing (1), the holes on the housing receiver and the front half of the light source housing (8) aligned such to allow light to pass through the bitewing (1). The bitewing (1) is then inserted into the mouth, the center of the bite surface (1) aligned with the center of the tooth to be assessed, and thus positioning the light source (5) on the lingual side and camera groove (2) on the opposite side of the targeted tooth. The patient then bites down on the bite surface, mechanically maintain alignment of the light source (5) and camera groove (2) in line with the center portion of the chosen tooth face. The intraoral camera head (image capturing device) is then placed onto the groove (2), and video capture is initiated. As shown in FIG. 4, light transmitted through the tooth is then captured and measured by the image capturing device (12). In an embodiment, additional lenses or filters (13) may be placed in front of the image capturing device (12) to reduce artifacts and noise. Light pass through the tooth thus adjusted to the desired wavelength before reaching the intraoral camera (5).

The light traveled through the tooth is then captured and measured by the image capturing device (12), which is operationally connected to a computing device (15). In one embodiment, the computing device (15) is equipped with customized PPG image processing software designed to provide near real-time PPG image analysis of captured sequential images (video) by integrating spatial decomposition, temporal filtration, amplification, and mapping through a single graphical user interface. The spatial decomposition serves to reduce spatial noise, and shrink the images in the spatial domain to reduce subsequent image processing times. Temporal bandpass filtering is then done in the time domain at each discrete spatial location within the images to filter out noise, and isolate pulsatile components within the frequency range of a typical heartbeat. The PPGI system may also incorporate a heartrate monitor that to adaptively adjust the temporal bandpass filter cutoff frequencies based on the heartrate of the patient. Once the pulsatile component of the signal is isolated, it is then amplified, and mapped so that spatial regions with greater pulsatility can be differentiated from regions with less pulsatility.

The spatiotemporal filtering and amplification parameters are optimized to maximize the contrast between images collected during the systolic and diastolic portions of the cardiac cycle. In an additional embodiment, the image processing software may further include the addition of an automatic pulp chamber selection tool for increased accuracy. Visual information provided by the tooth scan will also be investigated in further studies to determine the relationship of changes in pulse distribution with various pulpal conditions and tested with machine learning algorithms. In one embodiment, after the generation of a distribution map of pulpal tissue of the tooth, the dental clinician will use the Region of Interest (ROI) tool to highlight the parts of the tooth with the most movement. After choosing the RO, a pulse rate will be generated to assist the dental clinician or operator in making an assessment based on this direct measurement of vitality.

Compared to the current dental pulp vitality testing technologies, the PPGI-pulp vitality test technology provides a direct, cost-effective, radiation-free, non-invasive, and reliable diagnostic test of tissue oxygenation within a dental pulp. Pulp sensibility tests are the current gold-standard for assessing pulp vitality. The test assessments are indirect and subjective in nature, often generating false positive or negative results. Radiographs only provide images of the denser enamel and dentin regions of the tooth without any diagnostic utility for assessing the state of the pulp tissue. They also produce harmful ionizing radiation, often requiring specialized infrastructure and dedicated resources to operate and maintain.

Thus the PPGI-based pulp vitality test would provide a valuable diagnostic test capable of directly assessing the state of the pulp. The imaging system is expected, not only to identify the presence of blood flow in the pulp, but also to identify the regional disruptions or anastomosis caused by trauma or indicative of underlying pathology. The technology would be a low-cost, accessible, non-invasive, and provide rapid diagnostics in a small package that could be implemented in a clinic or a field-setting. The technique could improve upon the accuracy and reliability of current pulp vitality tests, to more readily identify pulp pathology, and reduce the number of potentially preventable endodontic dental emergencies occurring in-theater.

### Example 1: Construction of Prototype PPGI Based Pulp Vitality System

Imaging hardware and optics will be configured to measure light transmitted through the perfused tooth at discrete wavelengths in the visible-red and near-infrared spectrum. Image analysis software will be developed to process the images, and extract physiological characteristics associated with the pulse. The hardware configuration and imaging parameters will be optimized to maximize signal quality and resolution. A printed circuit board (PCB) located inside the bitewing was designed for production to operate the LED light source.

### Example 2: Testing of the PPGI Based Pulp Vitality System Using an Ex Vivo Perfused Tooth Model

Ex-vivo tooth model: A circulation system is designed to simulate pulp hemodynamics and oxygenation by pumping blood through the pulp chamber of extracted human molars. An oxygenator, which precisely controls blood oxygen saturation by promoting gas exchange between blood and a controlled mixture of oxygen, nitrogen, and carbon dioxide, will be used to condition reservoirs of blood representing both arterial and venous oxygenation saturation levels. Blood oxygen saturation in the arterial and venous reservoirs will be measured using blood gas analysis to confirm target the oxygenation. A dual dispensing pump will be used to alternate the flow of arterial and venous blood into the pulp to simulate changes in regional blood oxygenation during the cardiac cycle. An algorithm was developed to control the rates and volumes of the dual syringe pump, camera video acquisition, and gas flow during validation testing. The dual syringe pump control was used to draw, dispense, and circulate conditioned blood. The camera controls were used to record video from the camera and save the data in an audio video interleave (AVI) format for analysis using the PABLO algorithm. The gas controls were used before each experiment to introduce concentration of gases to the blood to condition it to appropriate arterial and venous oxygenation levels.

Materials and Methods: The PABLO algorithm is used to analyze the PPGI signals to determine dental pulp pulse frequency. Both the classification of a tooth as vital or nonvital and the pulse rate determination were made by quantifying the fluctuation of light intensity transmitted through the tooth during the cardiac cycle. A step-by-step demonstration is shown in FIG. 6, illustrating the PABLO algorithm graphical user interface (GUI). A) Video of ex vivo tooth illuminated by the LED was selected. B) PABLO GUI was used to load the video. C) A sequence of frames was extracted from the video. D) Feature detection and matching for video stabilization was performed. E) Grayscale video was transformed into an RGB colormap informing subsequent selection of ROI. F) Time and frequency domain analyses were generated to determine the pulse frequency of the pulp.

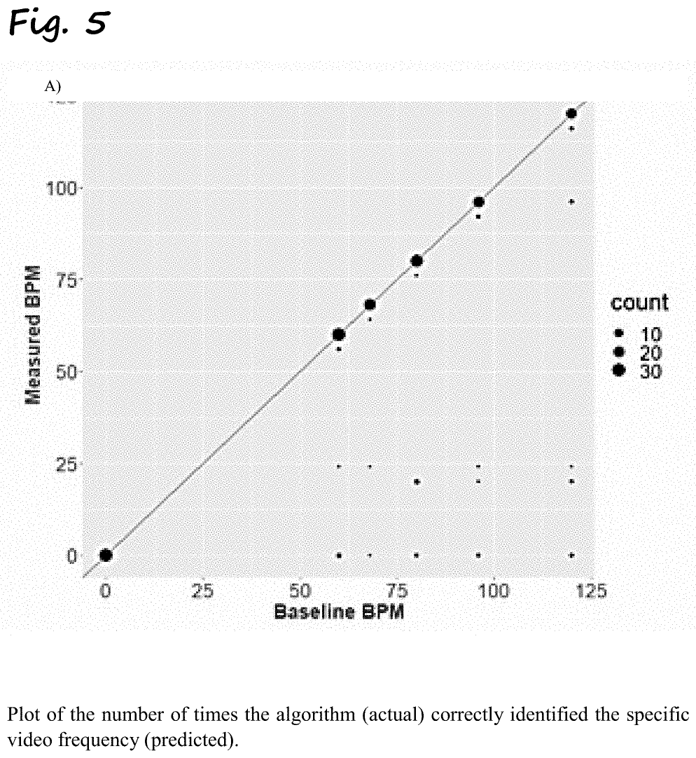

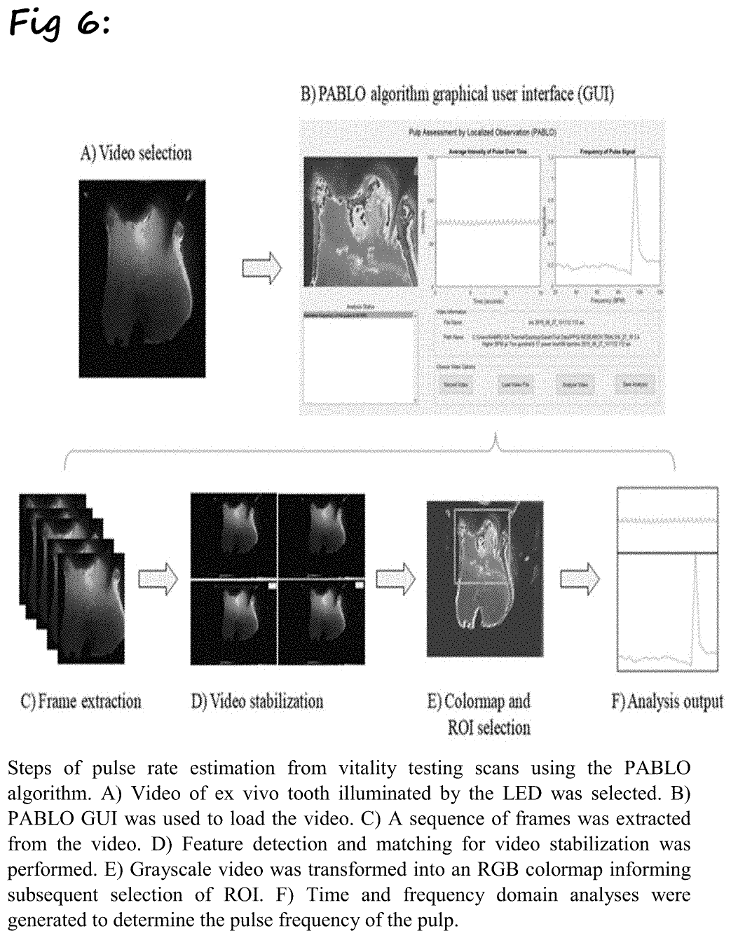

Results and Discussion: Results of the analysis (see FIG. 5 and Table 1) showed that the algorithm had a pulse detection sensitivity above 90% and a percent error less than 11% in all trials. In the control case, results showed a sensitivity of 93% and a pulse detection error of 7.3%, indicating that this algorithm has promise as a diagnostic tool for clinicians. The error rate was higher than pulse measurements produced by other commonly available vitality test systems, such as pulse oximetry. The specificity of the algorithm, 90%, decreased and the error, 11%, increased after the addition of a simulated gumline. These reductions were not found to be significant, however, demonstrating the ability of the algorithm to accurately identify the pulse even when the pulp chamber was partially obscured. Multiple teeth included in the camera's field of view were found to have no effect on the algorithm's accuracy or sensitivity of pulse detection, suggesting this algorithm's resistance to crosstalk from adjacent teeth in interpreting PPGI signals. With the stabilization algorithm applied in a situation with free operator-induced camera movement, no significant difference in errors between operation of the device in handheld mode and operation with the device immobilized was observed. The sensitivity and specificity values of the system were significantly higher than those of traditional pulpal diagnostic techniques, which are only reliable in about 80% of cases, indicating its ability to rival that of commonly used clinical techniques and highlighting its potential practicality in a clinical setting.

TABLE 1
Results of the PABLO analysis.

| | | Simulated | Adjacent Teeth | Video |
|---|---|---|---|---|
| | Control | Gumline | Interference | Stabilization |
| Sensitivity | 93.0% | 90.0% | 90.4% | 100.0% |
| Specificity | 100.0% | 79.4% | 94.4% | 100.0% |
| SMAPE | 7.3% | 11.0% | 9.6% | 6.3% |

The sensitivity, specificity, and symmetric mean absolute percent error (SMAPE) were calculated to understand how each confounding variable affected the algorithm's ability to detect and quantify pulse.

### Example 3: Automatic Determination of ROI Using Machine Learning

Future developments will incorporate machine learning algorithm to automatically determine ROI. The imaging system will be optimized by testing multiple camera-light configurations, and using wavelengths of interest throughout the visible-red and near-infrared spectrum, to maximize the strength and resolution of the measured signals. Simulations will be run with the teeth illuminated from either or both lingual and occlusal surfaces. Likewise, camera-position will be tested facing the buccal or occlusal surfaces. In each configuration, a series of wavelengths will be evaluated to identify those which exhibit the greatest signal strength, maximizing the contrast between images captured during the systolic and diastolic portion of the pulsatile waveform. During the initial testing, low venous oxygen saturations will be used to maximize the contrast between arterial and venous light absorption and serve as proof-of-principal for the technique. If visualization of pulp blood flow cannot be achieved in the maximum contrast condition, additional testing will not be perused. However, if the initial proof-of-principal testing is successful, the venous oxygen saturation will be elevated to physiologically normal levels for further system optimization and testing.

Future development efforts will also focus on further optimizing the image processing software to visualize the distribution of blood flow in real-time. The imaging technique will be evaluated across a range of different tooth morphologies in vivo and compared clinically against the predictive value of the gold-standard pulp sensibility tests. A long-term goal would be to use camera-based oximetry to monitor pulp disease progression over time, and identify metrics that could be used to identify the onset of underlying pathology. This novel technique could potentially provide valuable diagnostic information, enabling dentists to better assess the health of the pulp and guide endodontic treatment decisions.

### REFERENCES

### References—Updated List

[1] E. Chen and P. V. Abbott, “Dental Pulp Testing: A Review” Int. J. Dent. (2009) doi: 10.1155/2009/365785.
[2] V. Gopikrishna, G. Pradeep, and N. Venkateshbabu, “Assessment of pulp vitality: A review” Int. J. Paediatr. Dent. (2009) doi: 10.1111/j.1365-263X.2008.00955.x.
[3] J. J. Jespersen, J. Hellstein, A. Williamson, W. T. Johnson, and F. Qian, “Evaluation of Dental Pulp Sensibility Tests in a Clinical Setting” J. Endod. (2014) doi: 10.1016/j.joen.2013.11.009.
[4] A. Arun, H. Mythri, and D. Chachapan, “Pulp vitality tests—an overview on comparison of sensitivity and vitality” Indian J. Oral Sci. 6(2), 41 (2015) doi: 10.4103/0976-6944.162622.
[5] H. Jafarzadeh and P. V. Abbott, “Review of pulp sensibility tests. Part I: general information and thermal tests,” International Endodontic Journal. vols. 2010, doi: 10.1111/j.1365-2591.2010.01754.xp. no.vol.
[6] L. G. Levin, “Pulp and periradicular testing,” in Journal of Endodontics, 2013, doi: 10.1016/j.joen.2012.11.047.
[7] F. M. Andreasen and B. Kahler, “Diagnosis of acute dental trauma: The importance of standardized documentation: A review,” Dental Traumatology. vols. 2015, doi: 10.1111/edt.12187p. no.vol.
[8] J. Musselwhite, B. Klitzman, W. Maixner, and E. Burks, “Laser doppler flowmetry” Oral Surg Oral Med Oral Pathol Oral Radiol Endod 84(4), 411-419 (1997) doi: 10.1007/978-3-319-19327-4_9.
[9] H. Karayilmaz and Z. Kirzioglu, “Comparison of the reliability of laser Doppler flowmetry, pulse oximetry and electric pulp tester in assessing the pulp vitality of human teeth” J. Oral Rehabil. (2011) doi: 10.1111/j.1365-2842.2010.02160.x.
[10] K. Kijsamanmith, S. Timpawat, N. Vongsavan, and B. Matthews, “Pulpal blood flow recorded from human premolar teeth with a laser Doppler flow meter using either red or infrared light” Arch. Oral Biol. (2011) doi: 10.1016/j.archoralbio.2010.12.003.
[11] B. Anusha, K. Madhusudhana, S. K. Chinni, and Y. Paramesh, “Assessment of pulp oxygen saturation levels by pulse oximetry for pulpal diseases—a diagnostic study” J. Clin. Diagnostic Res. (2017) doi:10.7860/JCDR/2017/28322.10572.
[12] S. Polat, K. Er, and N. T. Polat, “Penetration depth of laser Doppler flowmetry beam in teeth” Oral Surgery, Oral Med. Oral Pathol. Oral Radiol. Endodontology (2005) doi: 10.1016/j.tripleo.2004.11.018.
[13] M. Bargrizan, M. A. Ashari, M. Ahmadi, and J. Ramezani, “The use of pulse oximetry in evaluation of pulp vitality in immature permanent teeth” Dent. Traumatol. (2016) doi: 10.1111/edt.12215.
[14] A. Niklas et al., “In vitro optical detection of simulated blood pulse in a human tooth pulp model” Clin. Oral Investig. (2014) doi: 10.1007/s00784-013-1115-z.
[15] V. Jain, B. P. Kumar, T. S. Krishna, S. Parakh, P. H. Kiran, and A. Tiwari, “Recent Advances in Endodontic Diagnosis: A Review” Int. J. Prev. Clin. Dent. Res. 4(4), 300-303 (2017) doi: 10.5005/jp-journals-10052-0130.
[16] S. Tewolde, J. Bequette, L. Martinez, and R. Dory, “Development of a Camera based System to Image the Distribution of Blood in Dental Pulp and Assess its Vitality,” in Military Health System Research Symposium, 2018.
[17] U. Rubins, V. Upmalis, O. Rubenis, D. Jakovels, and J. Spigulis, “Real-time photoplethysmography imaging system,” in IFMBE Proceedings, 2011, doi: 10.1007/978-3-642-21683-1_46.
[18] Cargill, W. H., and Hickam, J. B., The oxygen consumption of the normal and the diseased human kidney. J. Clin. Invest., 1949, 28, 526.
[19] R. J. Hyndman and A. B. Koehler, “Another look at measures of forecast accuracy” Int. J. Forecast. 22(4), 679-688 (2006) doi: 10.1016/j.ijforecast.2006.03.001.
[20] Demeulenaere, S. (2007). Pulse Oximetry: Uses and Limitations. The Journal for Nurse Practitioners, 3(5), 312-317. doi: 10.1016/j.nurpra.2007.02.021
[21] Deutsch, Allan & Musikant, Barry. Morphological Measurements of Anatomic Landmarks in Human Maxillary and Mandibular Molar Pulp Chambers. Journal of endodontics (2004). 30. 388-90. 10.1097/00004770-200406000-00003.
[22] J. E. Sinex, “Pulse oximetry: Principles and limitations” Am. J. Emerg. Med. 17(1), 59-66 (1999) doi:10.1016/50735-6757(99)90019-0.

What is claims is:

1) A system for assessing vitality of a tooth and measuring oxygenation and blood flow in pulpal tissue of said tooth comprising:
(a) a light source capable of emitting light that transilluminate said tooth;
(b) a bitewing;
(c) an image capturing device capable of detecting a plurality of sequential images of light transmitted through said tooth, wherein said image capturing device is hold in an aligned position with said light source on opposite sides of said tooth when a patient bite onto said bitewing;
(d) a computing device, which is operatively connected to said image capturing device and is equipped with an image processing software;
wherein said plurality of sequential image of said tooth are received and processed by said computing device to generate an distribution map of said tooth.

2) The system of claim 1, further comprising additional lenses or filters that are placed in front of the image capturing device to reduce artifacts and noise.

3) The system of claim 1, further comprising a heartrate monitor to adjust a temporal bandpass filter cutoff frequencies based on the heartrate of the patient.

4) The system of claim 1, wherein said light source is emitting light in the visible (350-780 nm) and near-infrared (780-2500 nm) spectrum.

5) The system of claim 4, wherein said light source comprises of one or more Light Emitting Diode (LED).

6) The system of claim 1, wherein said light source is powered by a wire power supply or a battery.

7) The system of claim 1, wherein said image capturing device is an intraoral camera.

8) The system of claim 1, wherein said computing device is a computer, a mobile computing device or a microchip.

9) The system of claim 8, wherein image processing software, is configured to generate a spectral signal at each pixel of the image of said tooth corresponding to the detected lights pass through said tooth over time, thus forming a distribution map of perfused pulpal tissue of said tooth.

10) The system of claim 9, wherein said system further comprise a display, showing distribution map of perfused pulpal tissue of said tooth.

11) The system of claim 1, further comprising a focusing device for focusing the light from the light source onto a measurement point on the tooth.

12) The system of claim 2, wherein said one or more filters separate the light passed through the tooth into selected wavelengths.

13) The system of claim 1, wherein said bitewing further include a means for mounting and aligning the light source, the focusing device, the lenses, the filter, and/or the camera on the side of the tooth to facilitate the transmission of light through the tooth and to the camera.

14) A method for real-time visualization and analysis of the vitality of the tooth, comprising:
a) Emitting light onto a surface of a tooth;
b) Capturing light passed through said tooth on the opposite surface of said tooth over a period of time to create a plurality of sequential images;
c) Processing said plurality of sequential image frames at each pixel to generate an distribution map of perfused pulpal tissues of said tooth, wherein a pulsed signal at a pixel of said sequential image frames represent perfused tissue, and static signal at said pixel represent non-perfused tissue; and
d) Assessing pulp vitality by analyzing displayed oxygenation distribution map of perfused pulpal tissues of said tooth.

15) The method of claim 14, further comprising the step of filtering out noise, and isolating pulsatile components within the frequency range of a typical heartbeat in said processing step by providing a heart rate monitor which produces a digital signal corresponding to the pulse of the patient heartbeat and using said digital signal in the processing step.

16) The method of claim 15, wherein said heartrate monitor is used to adaptively adjust a temporal bandpass filter cutoff frequencies based on the heartrate of the patient.

17) A method of claim 15, wherein said processing step further includes determining a synchronization between said signal and information from an ECG monitor.

18) An apparatus for taking a plurality of sequential images of a tooth for generating a distribution map of perfused tissues of a tooth, comprising a light source housing and a bitewing with a bite surface, a distal end and a proximal end, wherein said bitewing has a housing receiver in the distal end to receive a light source housing and a groove on periphery of the proximal end to position and hold an image capturing device; such that an one or more openings on the light source housing aligns with corresponding openings on the light source housing receiver to allow the passage of light from said light source housing through said tooth to said image capturing device.
